(19) United States
(12) Patent Application Publication
(10) Pub. No.: US 2023/0123099 A1
(43) Pub. Date: Apr. 20, 2023

(54) DEXTROSE TABLETS WITH IMPROVED MOUTHFEEL

(71) Applicant: FERTIN PHARMA A/S, Vejle (DK)

(72) Inventor: Helle WITTORFF, Vejle Ø (DK)

(21) Appl. No.: 17/502,322

(22) Filed: Oct. 15, 2021

(51) Int. Cl. A61K 9/00; A61K 9/20; A61K 45/06

(52) U.S. Cl. CPC A61K 9/0056; A61K 9/205; A61K 45/06

(57) ABSTRACT

The invention relates to an oral chewable tablet suitable for improved mouthfeel, comprising dextrose in an amount from 50 to 95% by weight of the tablet; one or more active ingredients; and one or more binders, wherein the ratio between the one or more binders and dextrose is from 1:250 to 1:8.

### TECHNICAL FIELD

The invention relates to the field of tablets for oral delivery of active ingredients. In particular, the tablets of the invention are suitable for high load delivery of active ingredients.

### BACKGROUND

Traditionally, tablets for delivery of active ingredients in the oral cavity have been applied in various forms and compositions. Common to most of the tablets in the prior art is that focus has mainly been on controlled release of the active ingredients or improvements with respect to taste-masking of the active ingredients since a major part of active ingredients are associated with bitterness.

Particularly relevant for tablets with a high load of active ingredients, challenges often arise during the manufacturing process. Often the high load of active ingredients provides fragile tablets that may cause processing problems in the manufacturing process, during storage and transportation of the tablets.

Also, a high load of active ingredients may cause issues for the overall sensorial properties of the tablets. Both the nature of the active ingredients with respect to taste properties and pronounced bitterness for some active ingredients, and the high amount of actives relative to other ingredients in the tablets, may be a challenge to formulation specialists.

One of the more critical issues is that it is often difficult to formulate tablets for oral delivery with suitable sensorial properties, particularly when the active ingredient is present in a high load. Sensorial properties in the present context include the overall mouthfeel of the tablet when chewed and the resulting delivery of the active ingredients in the oral cavity. For instance, a pronounced powdery sense is often not convenient to the user and as a result may often provide inferior tablets that are not used on a frequent basis.

Preferably, a formulation is to be provided that may also help in obtaining improved sensorics properties. Here, important sensorics properties include friability, texture, flavor perception, sweetness perception and off-notes associated with the actives. These properties are both relevant from a convenience perspective in chewable tablets, but certainly also in order to support an appropriate delivery of actives from the tablets and avoid adverse side effects of the actives.

One of the challenges with chewable tablets as a delivery vehicle of actives is that actives may tend to be associated with off-notes during administration due to the specific physiochemical properties of the compounds. The taste masking challenge is more profound when a higher release of the actives are delivered by such tablets. If off-notes are the predominant sensation during administration, convenience may be affected and even more critically, the delivery of the actives may also be affected. Saliva production may be suppressed, and the delivery vehicle may not be handled correctly.

Further, even when sensorial properties to some degree are complied with, there would usually also be a desire for relative fast delivery of active ingredients upon oral administration. Often, this desire of a relative fast release is counteracted by the desire for taste-masking of the active ingredients.

Particularly, less attention is given on the benefits of chewable tablet formulations that may help in obtaining a release characteristic of actives that offers increased convenience and effectiveness. One of these release characteristics is increased generation of saliva upon chewing. Increased saliva generation and particularly an experience of increased saliva generation upon administration may have some pronounced benefits for delivery of actives.

Accordingly, there is a need for tablets for oral delivery of active ingredients that both accommodate beneficial sensorial properties and at the same time accommodate a relative fast delivery of active ingredients. This may be particularly desired for active ingredients in a high load but may also be desired for active ingredients in a low load, such as ingredients with pronounced bitterness properties.

### SUMMARY

Accordingly, there is provided an oral chewable tablet suitable for improved mouthfeel, comprising

dextrose in an amount from 50 to 95% by weight of the tablet;

one or more active ingredients; and

one or more binders, wherein

the ratio between the one or more binders and dextrose is from 1:250 to 1:8.

Providing a chewable tablet according to the invention may solve various problems of the prior art and aims at establishing a chewable tablet that combines beneficial delivery properties of actives combined with advantageous sensorial properties.

Generally, the crux of the oral chewable tablets according to the invention is the combination of a relatively high amount of dextrose combined with one or more binders that are applied as separate elements in the formulation and one or more active ingredients. Hence, in the present context, the “one or more binders” are to be understood as binders that are added separately in the formulation and not being part or integrated in other ingredients in the formulation.

This combination of dextrose and one or more binders in the claimed ratio is believed to accommodate pronounced technical benefits to the present invention and is believed to allow for the presence of a high load of active ingredients while at the same time providing a chewable tablet that is less fragile and fulfills the desire of accommodating beneficial sensorial properties and a relative fast delivery of active ingredients.

One advantage of the invention is a surprisingly strong saliva generation compared to conventional chewable tablets and lozenges. Increased generation of saliva may have a huge impact on the delivery of the one or more active ingredients. Specifically, when increased generation of saliva is coordinated with release of the one or more active ingredients from the tablet, relatively quick delivery is obtained. Hence, a synergy between the effect of the one or more active ingredients and increased saliva generation may be seen according to the invention.

One unexpected advantage over the prior art is that the saliva generation is surprisingly sustained even after a user has swallowed the bulk-portion of the dextrose. This sustaining of the salivation generation may be advantageous in relation to many applications of a chewable tablet ranging from mouthfeel, taste, flavor perception, etc.

With respect to release properties, the present invention may offer an improved release profile of the one or more active ingredients compared to conventional chewable tablets. In particular, the specific tablet formulation platform of the present invention may serve to provide improved release characteristics of the one or more active ingredients compared to conventional chewable tablet formulation platforms applied in combination with one or more active ingredients.

A very important aspect of the present invention is the provision of beneficial sensorial properties. Here, important sensorial properties include mouthfeel, ease of chewing/melting into liquid, friability (mechanical robustness), texture, flavor perception, sweetness perception and off-notes associated with the one or more active ingredients. These properties are both relevant from a convenience perspective in chewable tablets, but certainly also in order to support an appropriate delivery of the one or more active ingredients from the chewable tablet formulation, such as an improved release profile, and to avoid adverse side effects of the one or more active ingredients.

The present inventor has shown very surprising results with the specific combination of features of the present invention in terms of these sensorial properties. It was an unexpected result that the invention could both contribute to an improved release profile, such as rapid release of the one or more active ingredients, and at the same time provide very beneficial sensorics properties which in terms may also support an appropriate delivery of the one or more active ingredients from the chewable tablet.

One of the sensorial properties that are particularly advantageous is friability of the oral tablet. Both in order to secure a desired release of the one or more active ingredients and to improve the sensation by a consumer, it is critical that friability is balanced. Also, the mouthfeel of the oral tablet during use is critical for the release of the one or more active ingredients and the experience as well as convenience during use. These properties may be improved by the present invention which was not expected by the inventor of the present invention.

In context of the present invention, a “chewable tablet” is intended to mean an oral tablet that is chewed upon oral administration, having characteristics allowing convenient chewing without adverse side effects associated with the texture of the oral tablet.

In some embodiments of the invention, the ratio between the one or more binders and dextrose is from 1:225 to 1:9. In some embodiments of the invention, the ratio between the one or more binders and dextrose is from 1:200 to 1:10.

Presently preferred is a content of about 1% of one or more binders in the chewable tablets according to the invention, such as a range about 0.5 to 3% of one or more binders in the chewable tablet according to the invention. Typically, this amount as well as the range may provide superior results for tablets with a content of 50 to 95% by weight of dextrose. Also typically, this amount may be beneficial both for tablets with a high of active ingredients, such as 30% load, or low load of active ingredients, such as 1 mg. While this amount is preferred, however, as will appear from further embodiments of the invention, other amounts or ranges may be applied with suitable results.

In some embodiments of the invention, the ratio between the one or more binders and dextrose is from 1:250 to 1:25 if the tablet comprises dextrose in an amount from 70 to 95% by weight of the tablet.

In the present context, the term “if the tablet comprises dextrose in an amount from 70 to 95% by weight of the tablet” or similar wordings is intended to be understood as a proviso that under the circumstances where the content of dextrose is in the range of 70 to 95% by weight of the tablet, then the ratio between the one or more binders and dextrose is from 1:250 to 1:25. This proviso is to be understood in context of the broader ratio between the one or more binders and dextrose and the broader range of the presence of dextrose, i.e., the proviso is a sub-division of the broader ratios and ranges.

Generally, in some embodiments, the higher the content of dextrose, the lower relative content of the one or more binders is required, and the lower the content of dextrose, the higher relative content of the one or more binders is required. Hence, the proviso illustrates this dynamic in the present invention for some embodiments.

In some embodiments of the invention, the ratio between the one or more binders and dextrose is from 1:250 to 1:50 if the tablet comprises dextrose in an amount from 70 to 95% by weight of the tablet.

In some further embodiments of the invention, the ratio between the one or more binders and dextrose is higher than 1:250 if the tablet comprises dextrose in an amount from 70 to 95% by weight of the tablet. In some further embodiments of the invention, the ratio between the one or more binders and dextrose is higher than 1:200 if the tablet comprises dextrose in an amount from 70 to 95% by weight of the tablet. In some further embodiments of the invention, the ratio between the one or more binders and dextrose is higher than 1:150 if the tablet comprises dextrose in an amount from 70 to 95% by weight of the tablet. In some further embodiments of the invention, the ratio between the one or more binders and dextrose is higher than 1:125 if the tablet comprises dextrose in an amount from 70 to 95% by weight of the tablet. In some further embodiments of the invention, the ratio between the one or more binders and dextrose is higher than 1:100 if the tablet comprises dextrose in an amount from 70 to 95% by weight of the tablet.

In some further embodiments of the invention, the ratio between the one or more binders and dextrose is lower than 1:25 if the tablet comprises dextrose in an amount from 70 to 95% by weight of the tablet. In some further embodiments of the invention, the ratio between the one or more binders and dextrose is lower than 1:50 if the tablet comprises dextrose in an amount from 70 to 95% by weight of the tablet. In some further embodiments of the invention, the ratio between the one or more binders and the dextrose is lower than 1:75 if the tablet comprises dextrose in an amount from 70 to 95% by weight of the tablet.

In some embodiments of the invention, the ratio between the one or more binders and dextrose is from 1:200 to 1:50 if the tablet comprises dextrose in an amount from 70 to 95% by weight of the tablet.

In some embodiments of the invention, the ratio between the one or more binders and dextrose is from 1:100 to 1:8 if the tablet comprises dextrose in an amount from 50 to 70% by weight of the tablet.

In the present context, the term “if the tablet comprises dextrose in an amount from 50 to 70% by weight of the tablet” or similar wordings is intended to be understood as a proviso that under the circumstances where the content of dextrose is in the range of 50 to 70% by weight of the tablet, then the ratio between the one or more binders and dextrose is from 1:100 to 1:8. This proviso is to be understood in context of the broader ratio between the one or more binders and dextrose and the broader range of the presence of dextrose, i.e., the proviso is a sub-division of the broader ratios and ranges.

Generally, in some embodiments, the higher the content of dextrose, the lower relative content of the one or more binders is required, and the lower the content of dextrose, the higher relative content of the one or more binders is required. Hence, the proviso illustrates this dynamic in the present invention for some embodiments.

In some embodiments of the invention, the ratio between the one or more binders and dextrose is from 1:100 to 1:10 if the tablet comprises dextrose in an amount from 50 to 70% by weight of the tablet.

In some embodiments of the invention, the ratio between the one or more binders and dextrose is from 1:75 to 1:15 if the tablet comprises dextrose in an amount from 50 to 70% by weight of the tablet.

In some further embodiments of the invention, the ratio between the one or more binders and dextrose is lower than 1:8 if the tablet comprises dextrose in an amount from 50 to 70% by weight of the tablet. In some further embodiments of the invention, the ratio between the one or more binders and dextrose is lower than 1:10 if the tablet comprises dextrose in an amount from 50 to 70% by weight of the tablet. In some further embodiments of the invention, the ratio between the one or more binders and dextrose is lower than 1:20 if the tablet comprises dextrose in an amount from 50 to 70% by weight of the tablet. In some further embodiments of the invention, the ratio between the one or more binders and dextrose is lower than 1:15 if the tablet comprises dextrose in an amount from 50 to 70% by weight of the tablet.

In some embodiments of the invention, the ratio between the one or more binders and the one or more active ingredients is from 1:75 to 1:8.

In some embodiments of the invention, the ratio between the one or more binders and the one or more active ingredients is from 1:50 to 1:10.

In some embodiments of the invention, the ratio between the one or more binders and the one or more active ingredients is from 1:30 to 1:15.

When a relatively low load of active ingredients are applied, the ratio between the one or more binders and the one or more active ingredients may be from 8:1 to 1:8. In some embodiments of the invention, the ratio between the one or more binders and the one or more active ingredients may be from 6:1 to 1:6. In some embodiments of the invention, the ratio between the one or more binders and the one or more active ingredients may be from 4:1 to 1:4. In some embodiments of the invention, the ratio between the one or more binders and the one or more active ingredients may be from 2:1 to 1:2.

In some further embodiments of the invention, the ratio between the one or more binders and the one or more active ingredients is higher than 1:250. In some further embodiments of the invention, the ratio between the one or more binders and the one or more active ingredients is higher than 1:200. In some further embodiments of the invention, the ratio between the one or more binders and the one or more active ingredients is higher than 1:150. In some further embodiments of the invention, the ratio between the one or more binders and the one or more active ingredients is higher than 1:125. In some further embodiments of the invention, the ratio between the one or more binders and the one or more active ingredients is higher than 1:100.

In some further embodiments of the invention, the ratio between the one or more binders and the one or more active ingredients is lower than 1:25. In some further embodiments of the invention, the ratio between the one or more binders and the one or more active ingredients is lower than 1:50. In some further embodiments of the invention, the ratio between the one or more binders and the one or more active ingredients is lower than 1:75.

In some further embodiments of the invention, the ratio between the one or more binders and the one or more active ingredients is lower than 1:8. In some further embodiments of the invention, the ratio between the one or more binders and the one or more active ingredients is lower than 1:10. In some further embodiments of the invention, the ratio between the one or more binders and the one or more active ingredients is lower than 1:20. In some further embodiments of the invention, the ratio between the one or more binders and the one or more active ingredients is lower than 1:15.

In some embodiments of the invention, dextrose is present in an amount from 55 to 95% by weight of the tablet. In some embodiments of the invention, dextrose is present in an amount from 60 to 95% by weight of the tablet. In some embodiments of the invention, dextrose is present in an amount from 65 to 95% by weight of the tablet. In some embodiments of the invention, dextrose is present in an amount from 70 to 95% by weight of the tablet. In some embodiments of the invention, dextrose is present in an amount from 70 to 90% by weight of the tablet.

In some embodiments of the invention, the oral chewable tablet is consisting essentially of dextrose, one or more active ingredients and one or more binders, except for auxiliary ingredients present up to about 5% by weight of the tablet.

Flavors, high intensity sweeteners and glidant are examples of auxiliary ingredients that may be added in low amounts according to the invention without compromising the platform consisting of dextrose, the one or more binders and the one or more active ingredients according to the invention.

However, in the present context, it is understood that a major part of the chewable tablets is composed of dextrose, the one or more binders and the one or more active ingredients according to the invention. The overall system of the chewable tablet is controlled by these ingredients, including the special improved sensorial benefits of the invention, such as an improved mouth-feel, as well as saliva generation, and as well as friability properties.

In some embodiments of the invention, the oral chewable tablet is consisting essentially of dextrose, one or more active ingredients and one or more binders.

In some embodiments of the invention, the oral chewable tablet does not comprise sugar alcohol. It is contemplated that sugar alcohols in some embodiments are detrimental to the platform properties according to the invention, including sensorial properties, such as mouth-feel. In some embodiments, however, low amounts of sugar alcohol may be added, such as low amount of mannitol, such as less than 5% by weight.

In some embodiments of the invention, the dextrose is based on controlled enzymatic hydrolysis of starch.

In some embodiments of the invention, the dextrose comprises anhydrous dextrose. In some embodiments of the invention, the dextrose comprises hydrated dextrose. In some embodiments of the invention, the dextrose comprises dextrose monohydrate.

In some embodiments of the invention, the dextrose comprises at least 90% dextrose equivalents calculated on a dry basis. In some embodiments of the invention, the dextrose comprises at least 93% dextrose equivalents calculated on a dry basis. In some embodiments of the invention, the dextrose comprises at least 95% dextrose equivalents calculated on a dry basis. In some embodiments of the invention, the dextrose comprises at least 98% dextrose equivalents calculated on a dry basis.

In some embodiments of the invention, the dextrose comprises a purified mixture of saccharides. In some embodiments of the invention, the dextrose comprises oligomeric saccharides. In some embodiments of the invention, the dextrose comprises 93 to 97% dextrose equivalents calculated on a dry basis. In some embodiments of the invention, the dextrose comprises microcrystalline dextrose. In some embodiments of the invention, the dextrose comprises dextrate.

In some embodiments of the invention, the dextrose does not comprise maltodextrin.

In some embodiments of the invention, the dextrose comprises 100% dextrose equivalents calculated on a dry basis. Dextrose in a pure version comprises 100% dextrose equivalents calculated on a dry basis and is presently preferred as the dextrose applied in the present invention. In some embodiments, dextrose is pure and is based on 100% conversion of starch to dextrose.

In some embodiments of the invention, the dextrose is directly compressible (DC).

In some embodiments of the invention, the dextrose is a powder.

In some embodiments of the invention, the dextrose comprises at least 30% by weight of particles in the range of 100 to 500 microns.

In some embodiments of the invention, the dextrose comprises at least 80% by weight of particles below 500 microns.

In some embodiments of the invention, the dextrose comprises at least 40% by weight of particles below 250 microns.

In some embodiments of the invention, the dextrose comprises at most 10% by weight of particles above 500 microns.

In some embodiments of the invention, the dextrose comprises at most 35% by weight of particles below 100 microns.

In some embodiments of the invention, the one or more binders is present in an amount of 0.4 to 5% by weight of the tablet.

In some embodiments of the invention, the one or more binders is present in an amount of 0.5 to 4% by weight of the tablet.

In some embodiments of the invention, the one or more binders is present in an amount of 0.7 to 3% by weight of the tablet.

In some embodiments of the invention, the one or more binders is present in an amount of 0.7 to 2% by weight of the tablet.

In some embodiments of the invention, the one or more binders is present in an amount of 0.7 to 1.3% by weight of the tablet.

In some embodiments of the invention, the one or more binders is selected from the group consisting of hydroxypropyl cellulose (HPC), hydroxypropyl methylcellulose (HPMC), and combinations thereof.

In some embodiments of the invention, the one or more binders comprise hydroxypropyl cellulose (HPC).

In some embodiments of the invention, the one or more binders is hydroxypropyl cellulose (HPC).

HPC may be applied as a particular attractive binder. Thus, this binder, when used with dextrose, exhibits an advantageous sensory experience when compared to other well-known binders. In particular, the use of HPC lower than 4% by weight of the tablet is advantageous, such as 0.1 to 3%, such as 0.1 to 2% by weight of the tablet.

In some embodiments of the invention, the one or more binders comprise hydroxypropyl methylcellulose (HPMC).

In some embodiments of the invention, the one or more binders is hydroxypropyl methylcellulose (HPMC).

HPMC may be applied as a particular attractive binder. Thus, this binder, when used with dextrose, exhibits an advantageous sensory experience when compared to other well-known binders. In particular, the use of HPMC lower than 4% by weight of the tablet is advantageous, such as 0.1 to 3%, such as 0.1 to 2% by weight of the tablet.

In some embodiments of the invention, the one or more binders does not comprise microcrystalline cellulose (MCC). It is contemplated that MCC is poor in relation to sensorial properties according to the invention.

In some embodiments of the invention, the one or more binders does not comprise silica, cellulose, silicified microcrystalline cellulose, clay, talc, starch, pregelatinized starch, calcium carbonate, dicalcium phosphate, magnesium carbonate, magnesium-alumino-metasilicates, hyper porous silica and mixtures thereof.

In some embodiments of the invention, the one or more active ingredients is present in an amount of 5 to 50% by weight of the tablet.

In some embodiments of the invention, the one or more active ingredients is present in an amount of 10 to 50% by weight of the tablet.

In some embodiments of the invention, the one or more active ingredients is present in an amount of 20 to 50% by weight of the tablet.

In some embodiments of the invention, the one or more active ingredients is present in an amount of 30 to 50% by weight of the tablet.

In some embodiments of the invention, the one or more active ingredients is present in an amount of 5 to 40% by weight of the tablet.

In some embodiments of the invention, the one or more active ingredients is present in an amount of 5 to 30% by weight of the tablet.

In some embodiments of the invention, the one or more active ingredients is present in an amount of 10 to 30% by weight of the tablet.

In some embodiments of the invention, the one or more active ingredients comprise a non-directly compressible (non-DC) active ingredient.

In some embodiments of the invention, the one or more active ingredients comprise a non-directly compressible (non-DC) active ingredient in an amount of 5 to 50% by weight of the tablet.

In some embodiments of the invention, the one or more active ingredients comprise a non-directly compressible (non-DC) active ingredient in an amount of 10 to 50% by weight of the tablet.

In some embodiments of the invention, the one or more active ingredients comprise a non-directly compressible (non-DC) active ingredient in an amount of 20 to 50% by weight of the tablet.

In some embodiments of the invention, the one or more active ingredients comprise a non-directly compressible (non-DC) active ingredient in an amount of 30 to 50% by weight of the tablet.

In some embodiments of the invention, the one or more active ingredients comprise a directly compressible (DC) active ingredient.

In some embodiments of the invention, the one or more active ingredients comprise an immune supporting active ingredient.

In some embodiments of the invention, the one or more active ingredients comprise a mixture of immune supporting active ingredients.

In some embodiments of the invention, the one or more active ingredients comprise a mixture of vitamins, minerals, and herbals.

In some embodiments of the invention, the one or more active ingredients comprise Vitamin C. In some embodiments of the invention, the one or more active ingredients comprise melatonin. In some embodiments of the invention, the one or more active ingredients comprise theanine. In some embodiments of the invention, the one or more active ingredients comprise calcium carbonate. In some embodiments of the invention, the one or more active ingredients comprise caffeine.

In some embodiments of the invention, the one or more active ingredients comprise multivitamin. In some embodiments of the invention, the one or more active ingredients comprise Zn-oxide. In some embodiments of the invention, the one or more active ingredients comprise Zn-citrate. In some embodiments of the invention, the one or more active ingredients comprise Zn-gluconate. In some embodiments of the invention, the one or more active ingredients comprise Vitamin D.

In some embodiments of the invention, the one or more active ingredients comprise acetaminophen. In some embodiments of the invention, the one or more active ingredients comprise phenylephrine. In some embodiments of the invention, the one or more active ingredients comprise dextromethorphan. In some embodiments of the invention, the one or more active ingredients comprise guaifenesin. In some embodiments of the invention, the one or more active ingredients comprise a combination of acetaminophen, phenylephrine, dextromethorphan, and guaifenesin.

In some embodiments of the invention, the one or more active ingredients comprise diphenhydramine. In some embodiments of the invention, the one or more active ingredients comprise loratadine. In some embodiments of the invention, the one or more active ingredients comprise nicotine.

In an embodiment of the invention the active ingredient is selected from acetylcysteine, ambroxol, amylmetacresol, benzocaine, bisacodyl, bismuth sub salicylate, bromhexine, cetirizine, dextromethorphan hydrobromide, 2,4-dichlorobenzyl alcohol, doxylamine succinate, flurbiprofen, glycerin, hexylresorcinol, lidocaine, menthol, myrrh, paracetamol, pectin, peppermint oil, phenol, phenylephrine hydrochloride, povidone-iodine, pseudoephedrine, ranitidine, simethicone, sodium docusate, spearmint, zinc, or any combination thereof.

The above list of active ingredients are active ingredients that may be delivered to the throat.

In some embodiments, the tablet may comprise further active ingredient, e.g. a combination of two or more active ingredients from the above list, or as a combination of an active ingredient from the above list and another active ingredient.

In an embodiment of the invention the active ingredient is an analgesic. Examples of analgesics include e.g. ibuprofen, paracetamol (acetaminophen), ketoprofen, aspirin (acetylsalicylic acid), and naproxen. In an embodiment of the invention the active ingredient is an anesthetic. In an embodiment of the invention the active ingredient is an anti-inflammation agent. In an embodiment of the invention the active ingredient is a disinfectant.

In an embodiment of the invention the active ingredient is a cough suppressant. Examples of cough suppressants include e.g. dextromethorphan.

In an embodiment of the invention the active ingredient is an expectorant, such as guaifenesin.

In an embodiment of the invention the active ingredient is a local anesthetic. Examples of local anesthetics include e.g. ambroxol, benzocaine and hexylresorcinol.

In an embodiment of the invention the active ingredient is a member of the morphinan class. Examples of members of the morphinan class include e.g. dextromethorphan.

In an embodiment of the invention the active ingredient is a non-steroidal anti-inflammatory drug (NSAID). Examples of non-steroidal anti-inflammatory drugs (NSAIDs) include e.g. flurbiprofen.

In an embodiment of the invention the active ingredient is an anti-inflammation agent. In an embodiment of the invention the active ingredient is a disinfectant.

In an embodiment of the invention the active ingredient is a cough and cold agent.

In an embodiment of the invention the tablet comprises cough and cold agents including acetaminophen, dextromethorphan hydrobromide, guaifenesin and phenylephrine hydrochloride.

In an embodiment of the invention the tablet comprises cough and cold agents including acetaminophen, dextromethorphan hydrobromide and phenylephrine hydrochloride.

In an embodiment of the invention the active ingredient is an antihistamine. In an embodiment of the invention the active ingredient is an antibiotic. Examples of antibiotics include e.g. ampicillin, erythromycin, tetracycline, clarithromycin, penicillin, and metronidazole.

In an embodiment of the invention the active ingredient is an enzyme. One advantage of enzymes may be that digestion may be accelerated and/or that intestinal balance is restored or improved. In an embodiment of the invention the active ingredient is an opioid.

In an embodiment of the invention the tablet is a medical device for alleviating or treating dysphagia by inducing saliva generation. In an embodiment of the invention the active ingredient is cetirizine. In an embodiment of the invention the active ingredient is bromhexine. In an embodiment of the invention the active ingredient is amylmetacresol. In an embodiment of the invention the active ingredient is paracetamol. In an embodiment of the invention active ingredient is acetaminophen. In an embodiment of the invention the active ingredient is dextromethorphan HBr. In an embodiment of the invention the active ingredient is guaifenesin. In an embodiment of the invention the active ingredient is phenylephrine HCl. In an embodiment of the invention the active ingredient is penicillin.

In an embodiment of the invention, the tablet further comprising a water-soluble fiber, such as inulin.

In an embodiment of the invention, the active ingredient comprises zinc gluconate and ascorbic acid.

In an embodiment of the invention, the tablet further comprising a plant extract, such as red clover or willow extract. In an embodiment of the invention, the tablet further comprising a plant extract, such as Echinacea, Camille or Lavender. In an embodiment of the invention, the plat extract is combined in the tablet with zinc gluconate and ascorbic acid.

In an advantageous embodiment of the invention the oral tablet is essentially consisting of ingredients that are present in nature.

In an advantageous embodiment of the invention the oral tablet comprises a natural high intensity sweetener, such as stevioside.

In some embodiments, the one or more active ingredients is selected from alginate, atenolol, aspirin (acetylsalicylic acid), ampicillin, aminosalicylates, anhydrous citric acid, aspirin, bisacodyl, bismuth subsalicylate, bupropion, caffeine, calcium, calcium carbonate, cetirizine, cimetidine, cisapride, clarithromycin, desloratadine, dexlansoprazole, diphenhydramine HCl, diphenhydramine citrate, dimenhydrinate, docusate erythromycin, dopamine, esomeprazole, famotidine, fexofenadine HCl, guaifenesin, hydrotalcite, ibuprofen, ketoprofen, lactase enzyme, lansoprazole, loratadine, lorcaserin, loperamide, loperamide HCl, magnesium, magnesium carbonate, magnesium hydroxide, melatonin, methamphetamine HCl, metoclopramide, metronidazole, montelukast, mycostatin, naltrexone, naproxen, naproxen sodium, nizatidine, omeprazole, ondansetron, orlistat, pantoprazole, paracetamol (acetaminophen), pectin, phentermine HCl, polypodium leucotomos, prednisolone, prednisone, progesterone, propranolol, propantheline bromide, pseudoephedrine HCl, phentermine, rabeprazole, ranitidine, roflumilast, scopoloamine butyl hydroxide, simethicone, sodium, sodium bicarbonate, sodium docusate, sumatriptan, testosterone, tetracycline, topiramate, vitamin A, vitamin B, vitamin B12, vitamin C (ascorbic acid), vitamin D, and vitamin E, vitamin K, prebiotics, probiotics, inulin fiber, citicoline, L-theanine, taurine, tryptophan, gamma-aminobutyric acid, or any combination thereof.

The above list of active ingredients are active ingredients that may be delivered to the gastrointestinal tract.

In some embodiments, the may comprise further active ingredient, e.g. a combination of two or more active ingredients from the above list, or as a combination of an active ingredient from the above list and another active ingredient.

Further ingredients include herbals, such as Ashwagandha, ginseng, elderberry, Boswellia, green tea, green coffee bean extract, coffee fruit extract, willow bark, Ivy leaf, rose hip, chamomile, forsythia fruit extract, lemon balm, passionflower extract, zembrin, and root of marshmallow.

In an advantageous embodiment of the invention the active ingredient is an analgesic. Examples of analgesics include e.g. ibuprofen, paracetamol (acetaminophen), ketoprofen, aspirin (acetylsalicylic acid), and naproxen. In an advantageous embodiment of the invention the active ingredient is an anesthetic. In an advantageous embodiment of the invention the active ingredient is an anti-inflammation agent. In an advantageous embodiment of the invention the active ingredient is a disinfectant. In an advantageous embodiment of the invention the active ingredient is an antibiotic. Examples of antibiotics include e.g. ampicillin, erythromycin, tetracycline, clarithromycin, penicillin, and metronidazole.

In an advantageous embodiment of the invention the active ingredient is selected from vitamins, minerals, and supplements (VMS).

Examples of vitamins, minerals, and supplements include e.g. vitamin A, vitamin B, vitamin B12, vitamin C, vitamin D, vitamin E, vitamin K.

In an advantageous embodiment of the invention the active ingredient is a hormone. In an advantageous embodiment of the invention the active ingredient is melatonin. Examples of hormones include e.g. progesterone, testosterone, and melatonin. In an advantageous embodiment of the invention the active ingredient is a steroid. Examples of steroids include e.g. prednisolone and prednisone. In an advantageous embodiment of the invention the active ingredient is a proton pump inhibitor. Examples of proton pump inhibitors include e.g. rabeprazole, pantoprazole, esomeprazole, dexlansoprazole, lansoprazole, and omeprazole.

In an advantageous embodiment of the invention the active ingredient is an antihistamine. Examples of antihistamines include e.g. cimetidine, ranitidine, famotidine, nizatidine, and desloratadine. Antihistamines are drugs to treat allergic rhinitis and other allergies. Antihistamines can give release to a person with nasal congestion, sneezing or hives caused by e.g. pollen, dust mites or animal allergy.

In an advantageous embodiment of the invention the active ingredient is a triptan. Examples of triptans include e.g. sumatriptan.

In an advantageous embodiment of the invention the active ingredient is a xerostomia mitigation agent, such as a xerostomia mitigation agent for cancer patients. In an advantageous embodiment of the invention the active ingredient is a migraine treatment agent. In an advantageous embodiment of the invention the active ingredient is an enzyme.

In an advantageous embodiment of the invention the active ingredient is a probiotic ingredient. In an advantageous embodiment of the invention the active ingredient is a prebiotic ingredient.

In an advantageous embodiment of the invention the active ingredient is a gastrointestinal medication. In this context a gastrointestinal medication is understood as an active ingredient acting in the gastrointestinal tract.

In an advantageous embodiment of the invention the active ingredient is an opioid. In an advantageous embodiment of the invention the active ingredient is an allergy medication. In an advantageous embodiment of the invention the active ingredient is loratadine. In an advantageous embodiment of the invention the active ingredient is diphenhydramine.

In an advantageous embodiment of the invention the tablet is a medical device for alleviating or treating dysphagia by inducing saliva generation.

In an advantageous embodiment of the invention the active ingredient is ampicillin. In an advantageous embodiment of the invention the active ingredient is ibuprofen. In an advantageous embodiment of the invention the active ingredient is ondansetron. In an advantageous embodiment of the invention the active ingredient is paracetamol (acetaminophen). In an advantageous embodiment of the invention the active ingredient is acetylsalicylic acid. In an advantageous embodiment of the invention the active ingredient is simethicone. In an advantageous embodiment of the invention the active ingredient is sodium docusate.

In some embodiments of the invention, the one or more active ingredients comprise an active pharmaceutical ingredient.

In some embodiments of the invention, friability of the tablet is less than 3%, such as less than 2%, such as less than 1.5%, wherein friability is measured according to European Pharmacopoeia 9.1, test method 2.9.7. by using a pharmaceutical friability-tester PTF 10E from Pharma Test.

In some embodiments of the invention, the tablet generates more than 1.5 mL saliva within 30 seconds from onset of mastication.

In some embodiments of the invention, the tablet generates more than 1.5 mL saliva within a period from 30 to 90 seconds from onset of mastication.

In some embodiments of the invention, the tablet generates more than 1.5 mL saliva within a period from 90 to 180 seconds from onset of mastication.

In some embodiments of the invention, the tablet generates more than 1.5 mL saliva within a period from 180 to 300 seconds from onset of mastication.

In some embodiments of the invention, the oral chewable tablet further comprises a saliva production inhibiting agent for controlling saliva production.

In some embodiments of the invention, the oral chewable tablet is designed to release the active ingredient in the oral cavity and designed to deliver a part of the active ingredient to the throat as part of saliva generated upon mastication of the tablet.

In some embodiments of the invention, the oral chewable tablet is designed to release the active ingredient in the oral cavity and designed to deliver a part of the active ingredient to the gastrointestinal tract as part of saliva generated upon mastication of the tablet.

In some embodiments of the invention, oral chewable tablet comprises means for accelerated release of the one or more active ingredient.

In some embodiments of the invention, the oral chewable tablet comprises one or more disintegrants operable to disintegrate the tablet within a period of 2 minutes or less in contact with oral saliva.

In the present context, “disintegrated” or “disintegrate” is intended to mean that the tablet is no longer to be considered a tablet but the tablet has been reduced and/or dispersed in saliva.

Specifically, the content of disintegrants greatly facilitate disintegration of the tablet according to the invention. However, while disintegrants have previously been used in tablet formulation science, the particular combination of disintegrants with dextrose according to the application would have been seen as problematic in view of the specific properties of dextrose. Various problems were suspected by the inventors of the present application, such as sensorial drawbacks and concentration issues with a high load of the active ingredients.

In some embodiments of the invention, the oral chewable tablet comprises one or more disintegrants selected from the group consisting of sodium croscarmellose, crospovidone, sodium starch glycolate, and combinations thereof.

In an embodiment of the invention, the one or more disintegrants comprises cross-linked polyvinylpyrrolidone.

In an embodiment of the invention, the one or more disintegrants comprises cross-linked polyvinylpyrrolidone and wherein at least 50% by weight of the cross-linked polyvinylpyrrolidone has a particle size below 50 micrometers.

In an embodiment of the invention, the one or more disintegrants comprises cross-linked polyvinylpyrrolidone and wherein at least 25% by weight of the cross-linked polyvinylpyrrolidone has a particle size below 15 micrometers.

In some embodiments of the invention, the oral chewable tablet in contact with saliva has a disintegration profile that varies less than 10% under a compression pressure of 10 to 30 kN.

In some embodiments of the invention, the unit weight of the tablet is from about 100 mg to about 2000 mg. In some embodiments of the invention, the unit weight of the tablet is from about 100 mg to about 1800 mg. In some embodiments of the invention, the unit weight of the tablet is from about 500 mg to about 1600 mg. In some embodiments of the invention, the unit weight of the tablet is from about 600 mg to about 1500 mg.

In some embodiments of the invention, wherein the one or more active ingredients are present in an amount of 1 to 1000 mg. In some embodiments of the invention, wherein the one or more active ingredients are present in an amount of 1 to 800 mg. In some embodiments of the invention, the one or more active ingredients are present in an amount of 1 to 600 mg.

In some embodiments of the invention, the one or more active ingredients are present in an amount of 50 to 250 mg. In some embodiments of the invention, the one or more active ingredients are present in an amount of 100 to 250 mg.

In some embodiments of the invention, the one or more active ingredients are present in an amount of 1 to 50 mg. In some embodiments of the invention, the one or more active ingredients are present in an amount of 1 to 40 mg. In some embodiments of the invention, the one or more active ingredients are present in an amount of 1 to 30 mg. In some embodiments of the invention, the one or more active ingredients are present in an amount of 1 to 20 mg. In some embodiments of the invention, the one or more active ingredients are present in an amount of 1 to 10 mg. In some embodiments of the invention, the one or more active ingredients are present in an amount of 1 to 5 mg. In some embodiments of the invention, the one or more active ingredients are present in an amount of 1 to 4 mg.

In some embodiments of the invention, the oral chewable tablet provides an improved mouthfeel compared to an oral chewable tablet comprising less than 50% by weight of dextrose.

In some embodiments of the invention, the oral chewable tablet provides an improved melting sensation compared to an oral chewable tablet comprising less than 50% by weight of dextrose.

In some embodiments of the invention, the oral chewable tablet provides an improved liquid sensation compared to an oral chewable tablet comprising less than 50% by weight of dextrose.

In some embodiments of the invention, the oral chewable tablet provides a less stickiness sensation compared to an oral chewable tablet comprising less than 50% by weight of dextrose.

In some embodiments of the invention, the oral chewable tablet provides a less bitterness sensation from the one or more active ingredients compared to an oral chewable tablet comprising less than 50% by weight of dextrose.

In some embodiments of the invention, the oral chewable tablet provides improved taste masking compared to an oral chewable tablet comprising less than 50% by weight of dextrose.

In some embodiments of the invention, the oral chewable tablet provides improved friability compared to an oral chewable tablet that does not comprise one or more binders.

In some embodiments of the invention, the oral chewable tablet is designed to turn into liquid within 60 seconds of mastication.

In some embodiments of the invention, the oral chewable tablet is designed to turn into liquid within 30 seconds of mastication.

In some embodiments of the invention, the oral chewable tablet is designed to turn into liquid within 15 seconds of mastication.

In some embodiments of the invention, the oral chewable tablet comprises a further tablet module that is different in composition.

According to an embodiment of the invention, the tablet has two modules. Optionally, a coating may be applied around the two modules to form the final tablet.

In an embodiment of the invention, the chewable tablet comprises at least two modules. A tablet comprising two or more modules will have module sizes which each are comparable to the volume of the complete tablet. Comparable in the present context means that the modules are not understood as small particles and a module should at least be greater than 1/20 of the complete tablet volume, preferably greater than 1/10 of the complete tablet volume.

The module may typically be gathered from a plurality of compressed particles and have a weight which is greater than 0.2 gram and less than 10 grams.

In an embodiment of the invention, a module is defined as a plurality of particles being compressed together to form a gathered module of particles.

In an embodiment of the invention, the oral tablet comprises a plurality of oral tablet modules. In the present context the application of e.g., two modules are in particular advantageous as the use of dextrose may result in a more fragile tablet or at least the module in which the dextrose is present. In other words, dextrose may be present primarily in one module thereby optimizing the desired salivation and sensory experience from the module and the tablet as such whereas another module may serve as a support ensuring that the desired stability and friability of the complete tablet is obtained.

In an embodiment of the invention, the plurality of modules are slice-like layers. The term “slice-like” layer is a plain but very precise way of to the skilled person how a module may be provided, as such a layer is a standard structure obtained through conventional tableting procedures.

An advantage of using two modules is described above, but it should also be noted that this effect may also be obtained when applying layers of very different nature. Such application may e.g. include the use of a gum module and a non-gum module, where the non-gum modules are containing the dextrose particles. In this way, the non-gum layer may release the advantageous dextrose and the gum layer may both stabilize the tablet as described above but also interact with the dextrose during in particular the initial release for establishment of a very pleasant and impressing initial chew phase. This includes an increased saliva and moisture experience.

In an embodiment of the invention said population of particles is tableted into a first module and combined with a second population of particles that is tableted into a second module, where the second population of particles is different from the first population of particles.

In an embodiment of the invention, the dextrose is evenly distributed in the tablet or at least one module of the tablet.

One advantage of the above embodiment may be that the even distribution of the dextrose promotes an effective disintegration of the module upon mastication, e.g., due to lower mechanical strength contribution from the dextrose, thereby facilitating effective contacting of the resulting mastication fragments formed by the mastication with saliva, again increasing dissolving of the tablet. Also, the even distribution of the dextrose promotes a high number of mastication fragments with dextrose, which again effectively promotes salivation. Thus, a synergy between utilization of dextrose as a disintegration promoter due to the lower mechanical strength and also as a salivation promoter in combination with the even distribution to facilitate effect dispersion of mastication fragments in the oral cavity upon mastication.

In some embodiments of the invention, the oral chewable tablet comprises a further tablet module with a different disintegration time.

In an embodiment of the invention, the resistance to crunching of the tablet is greater than 60N, such as greater than 70N, such as greater than 80N, such as greater than 90N, such as greater than 100 N, such as greater than 110, such as greater than 130N such as greater than 150N, wherein the resistance to crunching of the tablet is less than 300N, such as less than 250N, such as less than 200N, wherein the resistance to crunching is determined according to European Pharmacopoeia 9.1, test method 2.9.8. by using a pharmaceutical resistance to crunching tester model Pharma Test type PTB 311.

High intensity artificial sweetening agents can also be used alone or in combination with the above dextrose. Preferred high intensity sweeteners include, but are not limited to sucralose, aspartame, salts of acesulfame, alitame, saccharin and its salts, cyclamic acid and its salts, glycyrrhizin, dihydrochalcones, thaumatin, monellin, stevioside (natural intensity sweetener) and the like, alone or in combination. In order to provide longer lasting sweetness and flavor perception, it may be desirable to encapsulate or otherwise control the release of at least a portion of the artificial sweeteners. Techniques such as wet granulation, wax granulation, spray drying, spray chilling, fluid bed coating, conservation, encapsulation in yeast cells and fiber extrusion may be used to achieve desired release characteristics. Encapsulation of sweetening agents can also be provided using another tablet component such as a resinous compound.

Usage level of the artificial sweetener will vary considerably and will depend on factors such as potency of the sweetener, rate of release, desired sweetness of the product, level and type of flavor used and cost considerations. Thus, the active level of artificial sweetener may vary from about 0.001 to about 3% by weight (preferably from about 0.02 to about 3% by weight). When carriers used for encapsulation are included, the usage level of the encapsulated sweetener will be proportionately higher. Combinations of sugar and/or non-sugar sweeteners may be used in the formulation.

In an embodiment of the invention, the tablet comprises flavor.

The amount of flavor may e.g., be from 0.1 to about 5% by weight of the tablet, such as 0.1 to about 3% by weight of the tablet.

Usable flavors include almond, almond amaretto, apple, Bavarian cream, black cherry, black sesame seed, blueberry, brown sugar, bubblegum, butterscotch, cappuccino, caramel, caramel cappuccino, cheesecake (graham crust), chili, cinnamon redhots, cotton candy, circus cotton candy, clove, coconut, coffee, clear coffee, double chocolate, energy cow, ginger, glutamate, graham cracker, grape juice, green apple, Hawaiian punch, honey, Jamaican rum, Kentucky bourbon, kiwi, koolada, lemon, lemon lime, tobacco, maple syrup, maraschino cherry, marshmallow, menthol, milk chocolate, mocha, Mountain Dew, peanut butter, pecan, peppermint, raspberry, banana, ripe banana, root beer, RY 4, spearmint, strawberry, sweet cream, sweet tarts, sweetener, toasted almond, tobacco, tobacco blend, vanilla bean ice cream, vanilla cupcake, vanilla swirl, vanillin, waffle, Belgian waffle, watermelon, whipped cream, white chocolate, wintergreen, amaretto, banana cream, black walnut, blackberry, butter, butter rum, cherry, chocolate hazelnut, cinnamon roll, cola, creme de menthe, eggnog, English toffee, guava, lemonade, licorice, maple, mint chocolate chip, orange cream, peach, pina colada, pineapple, plum, pomegranate, pralines and cream, red licorice, salt water taffy, strawberry banana, strawberry kiwi, tropical punch, tutti frutti, vanilla, or any combination thereof.

In an advantageous embodiment of the invention, said population of particles is tableted into a first module and combined with a second population of particles that is tableted into a second module.

In an advantageous embodiment of the invention, the tablet comprises particles comprising gum base.

In an advantageous embodiment of the invention the dextrose, one or more binders and one or more active ingredients are comprised in a first module and particles comprising gum base is comprised in a second module.

Thus, the oral tablet comprises a first module and a second module, the first module comprising the dextrose, one or more binders and one or more active ingredients, the second module comprising particles comprising gum base.

In an advantageous embodiment of the invention the dextrose, one or more binders and one or more active ingredients are tableted into a first module and particles comprising gum base is tableted into a second module, wherein the first module is free of gum base.

In an embodiment of the invention, the particles comprising gum base have an average particle size of at least 400 μm, such as between 400 μm and 1400 μm.

According to an embodiment of the invention, the particles comprising gum base consists of gum base. When the gum base particles consist of gum base, they typically have an average particle size between 800 μm and 1400 μm.

In an embodiment of the invention, the tablet comprises at least 20% by weight of gum base in a second module.

In an embodiment of the invention the oral tablet comprises between 20% and 60% by weight of gum base in a second module.

In an embodiment of the invention, the tablet is free of gum base.

In an embodiment of the invention, the product is a powder being compressed to a chewable tablet.

### DETAILED DESCRIPTION

The invention will now be described in more details with respect to certain aspects and embodiments of the invention. These aspects and embodiments are intended to be understood in connection with the rest of the description, including the Summary of the Invention and the Examples of the invention.

The verb “to comprise” as is used in this description and in the claims and its conjugations are used in its non-limiting sense to mean that items following the word are included, but items not specifically mentioned are not excluded. In addition, reference to an element by the indefinite article “a” or “an” does not exclude the possibility that more than one of the elements are present, unless the context clearly requires that there is one and only one of the elements. The indefinite article “a” or “an” thus usually means “at least one”. Additionally, the words “a” and “an” when used in the present document in connection with the word comprising or containing denote “one or more.” The expression “one or more” is intended to mean one, two, three or more.

As used herein, the term “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

As used herein the term “oral chewable tablet” is considered a tablet for oral use. Particularly, the oral tablet is considered as formed by tableting, i.e., compression of a particle composition, comprising a population of particles. Thus, the tablet is considered a compressed tablet formed by a plurality of particles. Typically, the oral chewable tablet may also be referred to as a tablet or oral tablet.

The term “particle size” relates to the ability of the particles to move through or be retained by sieve holes of a specific size. As used herein, the term “particle size” refers to the average particle size as determined according to European Pharmacopoeia 9.1 when using test method 2.9.38 particle size distribution estimation by analytical sieving, unless otherwise specifically is mentioned.

The term “particle” or similar wording is intended to denote a single, discrete composition of solid matter, such as a granule or individual elements in powder, having a certain size that may deviate considerable.

The term “weight of the oral tablet” or similar wording meaning the same is defined in the present context as weight of the oral tablet, not including the weight of an outer coating, such as a hard coating, soft coating, and the like.

By the phrase “texture” is meant a qualitative measure of the properties of the oral tablet and of the overall mouth-feel experienced by the user during use. Thus, the term “texture” encompasses measurable quantities such as hardness as well as more subjective parameters related to the feel experienced by a user.

The term “release” in the present context is intended to mean under “in vitro” conditions if not stated otherwise. In particular, the “release rate” during a certain period of time is intended to mean the amount in percentage of active ingredients that is released during the period. In the present context the term “release” refers to the released substance being liberated from the water-soluble matrix. In some embodiments, the process of releasing a substance corresponds to the substance being dissolved in saliva.

The term “sustained release” or “extended release” is herein intended to mean prolonged release over time. The term “rapid release” or “quick release” or “high release” is herein intended to mean a higher content released for a given period of time. The term “controlled release” is intended to mean a release of a substance from an oral tablet by the aid of active use of the oral tablet in the oral cavity of the subject, whereby the active use is controlling the amount of substance released.

In the present context the term “turn into liquid” is intended to mean that the tablet disintegrates and the fragments or particles of the tablet are either suspended or dissolved in saliva, perceived as liquid by a test person.

As used herein, the term “disintegrate” refers to a reduction of an object to components, fragments or particles. Disintegration time may be measured in vitro or in vivo. Unless otherwise stated, the in vitro measurements are carried out in accordance to European Pharmacopeia 9.0, section 2.9.1, Disintegration of tablets and capsules.

As used herein, the term “dissolve” is the process where a solid substance enters a solvent (oral saliva) to yield a solution. Unless otherwise stated, dissolving implies a full dissolving of the compound in question.

As used herein, the terms “disintegrant” refers to an ingredient facilitating disintegration of an FDT-module, when the FDT-module comes into contact with saliva. Disintegrants usable within the scope of the invention may include starch, pregelatinated starch, modified starch (including potato starch, maize starch, starch 1500, sodium starch glycolate and starch derivatives), cellulose, microcrystalline cellulose, alginates, and superdisintegrants, such as crosslinked cellulose (such as sodium carboxy methyl cellulose), crosslinked polyvinyl pyrrolidone (PVP), crosslinked starch, crosslinked alginic acid, natural superdisintegrants, and calcium silicate. Disintegrants may often be considered as measure promoting the break-up of the module into smaller fragments upon administration to facilitate nicotine release and eventual absorption. Crospovidone may comprise various grades, such as Kollidon CL-F or Kollidon CL-SF available from BASF.

When referring to induced saliva generation, it is noted that this induced saliva generation exceeds any saliva generation without the use of the tablet of the invention, or with a content of less than 50% dextrose. Particularly, in an embodiment, the induced saliva generation exceeds saliva generation when using conventional tablets without dextrose or with less than 50% dextrose. Then, induced saliva generation is increased over any saliva generation associated with conventional products, e.g. by comparing with a tablet without dextrose or with less than 50% dextrose.

When referring to induced saliva generation, the saliva generation may be tested using the following method:

Test subject abstain from eating and drinking at least 30 minutes before initiation of any test. Immediately before introducing of the tablet into the oral cavity, the test subject swallows. The test subject refrains from swallowing during the test. Immediately after introducing of the tablet into the oral cavity, the test subject starts masticating the tablet at a frequency of 1 chew per second for 20 seconds. Then, saliva and any remains of the tablet is kept in the mouth within chewing for 10 second. 30 seconds after starting the test, the test subject discards saliva including any tablet fragments into a plastic cup, which is weighted. Saliva discarded also at 90 seconds after onset of mastication, at 180 seconds after onset of mastication, at 300 seconds after onset of mastication, at 420 seconds after onset of mastication, and at 600 seconds after onset of mastication. At all times, the test subject makes as little movement as possible, and refrains from swallowing.

As used herein the term “active ingredient” refers to a substance that is biologically active and has a physiological effect on the human body for the benefit of the human body or part thereof. Active ingredients include active pharmaceutical ingredients, but also other active substances such as nutraceuticals or immune supporting active ingredients.

In the following raw materials will refer to the mixed particles to be compressed into a tablet according to embodiments of the invention unless otherwise stated.

The following description outlines explanations of how the tablet of the invention may be produced and further details of what may be added to the inventive composition.

Typically, the process of manufacture of the inventive tablet may be performed in a single tablet press, such as a rotary tablet press. But it may be a benefit under some circumstances to apply a separate tablet press.

Preferably, the upper punch is convex which gives the upper face of the pressed tablet a concave form.

It should of course be noted that the shape of the punches may vary depending on the desired tablet shape.

In some embodiments of the invention, pressing of the tablets are performed at a force of 10 to 50 kN. In some embodiments of the invention, pressing of the tablets are performed at a force of 10 to 40 kN. In some embodiments of the invention, pressing of the tablets are performed at a force of 10 to 30 kN.

The oral tablet according to the invention is manufactured by applying pressure to a content of particles by suitable compression means. The particles or powder is then pressed into a compact coherent tablet. The particles may for example comprise so-called primary particles or aggregated primary particles. When these are pressed, bonds are established between the particles or granules, thereby conferring a certain mechanical strength to the pressed tablet.

It should be noted that the above-introduced terms: powder, primary particles and aggregated primary particles may be somewhat misleading in the sense that the difference between primary particles and aggregated primary particles may very often be looked upon differently depending on the background of the user. Some may for instance regard a sweetener as a primary particle in spite of the fact that this particle due to the typically preprocessing performed when delivered to the customer should rather be regarded as some sort of aggregated primary particles. The definition adopted in the description of this invention is that aggregated primary particles refer to macro-particles comprising more or less preprocessed primary particles.

When pressure is applied to the particles, the bulk volume is reduced, and the amount of air is decreased. During this process energy is consumed. As the particles come into closer proximity to each other during the volume reduction process, bonds may be established between the particles or granules. The formation of bonds is associated with a reduction in the energy of the system as energy is released. Volume reduction takes place by various mechanisms and different types of bonds may be established between the particles or granules depending on the pressure applied and the properties of the particles or granules. The first thing that happens when a powder is pressed is that the particles are rearranged under low compaction pressures to form a closer packing structure. Particles with a regular shape appear to undergo rearrangement more easily than those of irregular shape. As the pressure increases, further rearrangement is prevented, and subsequent volume reduction is obtained by plastic and elastic deformation and/or fragmentation of the tablet particles. Brittle particles are likely to undergo fragmentation, i.e. breakage of the original particles into smaller units. Plastic deformation is an irreversible process resulting in a permanent change of particle shape, whereas the particles resume their original shape after elastic deformation. Evidently, both plastic and elastic deformation may occur, when compressing an oral tablet.

Several studies of the bond types in pressed tablets have been made over the years, typically in the context of pharmaceuticals and several techniques of obtaining pressed tablets on the basis of available powders has been provided. Such studies have been quite focused on what happens when the volume reduction is performed and how the end-product may be optimized for the given purpose. Several refinements with respect to pressed tablets has for instance been made in the addition of for example binders in the tablet raw materials for the purpose of obtaining a sufficient strength to the final pressed tablet while maintaining acceptable properties, e.g. with respect to release.

By the method of the invention, it is possible to form one-layered or multi-layered tablets, such as two-layered tablets or three-layered tablets.

In accordance with the invention, the tableted oral tablet according to the invention may comprise about 0.1 to about 75% by weight of an outer coating applied onto the oral tablet centre. Thus, suitable coating types include hard coatings, film coatings and soft coatings of any composition including those currently used in coating of tableted oral tablet.

One presently preferred outer coating type is a hard coating, which term is used in the conventional meaning of that term including sugar coatings and sugar-free (or sugarless) coatings and combinations thereof. The object of hard coating is to obtain a sweet, crunchy layer, which is appreciated by the consumer and it may moreover protect the oral tablet centres for various reasons. In a typical process of providing the oral tablet centres with a protective sugar coating, the oral tablet centres are successively treated in suitable coating equipment with aqueous solutions of crystallisable sugar such as sucrose or dextrose, which, depending on the stage of coating reached, may contain other functional ingredients, e.g. fillers, binding agents, colours, etc. In the present context, the sugar coating may contain further functional or active compounds including flavour compounds and/or active compounds.

In a typical hard coating process as it will be described in detail in the following, a suspension containing crystallisable sugar and/or polyol is applied onto the oral tablet centres and the water it contains is evaporated off by blowing with air. This cycle must be repeated several times, typically 3 to 80 times, in order to reach the swelling required. The term “swelling” refers to the increase in weight or thickness of the products, as considered at the end of the coating operation by comparison with the beginning, and in relation to the final weight or thickness of the coated products. In accordance with the present invention, the coating layer constitutes about 0.1 to about 75% by weight of the finished oral tablet element, such as about 10 to about 60% by weight, including about 15 to about 50% by weight.

In further useful embodiments, the outer coating of the oral tablet element of the invention is an element that is subjected to a film coating process and which therefore comprises one or more film-forming polymeric agents and optionally one or more auxiliary compounds, e.g. plasticizers, pigments and opacifiers. A film coating is a thin polymer-based coating applied to an oral tablet centre of any of the above forms. The thickness of such a coating is usually between 20 and 100 μm.

Generally, the film coating is obtained by passing the oral tablet centres through a spray zone with atomized droplets of the coating materials in a suitable aqueous or organic solvent vehicle, after which the material adhering to the oral tablet centres is dried before the next portion of coating is received. This cycle is repeated until the coating is complete.

In one embodiment the tablet according to the invention comprises a pharmaceutically, cosmetically or biologically active substance. Examples of such active substances, a comprehensive list of which is found e.g. in WO 00/25598, which is incorporated herein by reference, include drugs, dietary supplements, antiseptic agents, pH adjusting agents, anti-smoking agents. Examples of useful active substances in the form of antiseptics include salts and derivatives of guanidine and biguanidine the following types of substances with limited water-solubility: quaternary ammonium compounds (e.g. ceramine, chloroxylenol, crystal violet, chloramine), aldehydes (e.g. paraformaldehyde), derivatives of dequaline, polynoxyline, phenols (e.g. thymol, p-chlorophenol, cresol), hexachlorophene, salicylic anilide compounds, triclosan, halogenes (iodine, iodophores, chloroamine, dichlorocyanuric acid salts), alcohols (3,4 dichlorobenzyl alcohol, benzyl alcohol, phenoxyethanol, phenylethanol), cf. also Martindale, The Extra Pharmacopoeia, 28th edition, pages 547-578; metal salts, complexes and compounds with limited water-solubility, such as aluminum salts, (for instance aluminum potassium sulphate A1K(SO4)2, 12H2O) and salts, complexes and compounds of boron, barium, strontium, iron, calcium, zinc, (zinc acetate, zinc chloride, zinc gluconate), copper (copper chloride, copper sulphate), lead, silver, magnesium, sodium, potassium, lithium, molybdenum, vanadium should be included.

Examples of active substances in the form of agents adjusting the pH in the oral cavity include: acids, such as adipic acid, succinic acid, fumaric acid, or salts thereof or salts of citric acid, tartaric acid, malic acid, acetic acid, lactic acid, phosphoric acid and glutaric acid and acceptable bases, such as carbonates, hydrogen carbonates, phosphates, sulphates or oxides of sodium, potassium, ammonium, magnesium or calcium, especially magnesium and calcium.

Active ingredients may comprise the below mentioned compounds or derivates thereof but are not limited thereto: Acetaminophen, Acetylsalicylic acid, Buprenorphine, Bromhexin, Celcoxib, Codeine, Diphenhydramin, Diclofenac, Etoricoxib, Ibuprofen, Indometacin, Ketoprofen, Lumiracoxib, Morphine, Naproxen, Oxycodon, Parecoxib, Piroxicam, Pseudoefedrin, Rofecoxib, Tenoxicam, Tramadol, Valdecoxib, Calciumcarbonat, Magaldrate, Disulfiram, Bupropion, Nicotine, Azithromycin, Clarithromycin, Clotrimazole, Erythromycin, Tetracycline, Granisetron, Ondansetrone, Prometazin, Tropisetron, Brompheniramine, Ceterizin, leco-Ceterizin, Chlorcyclizine, Chlorpheniramin, Chlorpheniramin, Difenhydramine, Doxylamine, Fenofenadin, Guaifenesin, Loratidin, des-Loratidin, Phenyltoloxamine, Promethazin, Pyridamine, Terfenadin, Troxerutin, Methyldopa, Methylphenidate, Benzalcon. Chloride, Benzeth. Chloride, Chloride, Ecabet-sodium, Haloperidol, Allopurinol, Colchinine, Theophylline, Propanolol, Prednisolone, Prednisone, Urea, Actot, Glibenclamide, Glipizide, Metformin, Miglitol, Repaglinide, Rosiglitazone, Apomorfin, Cialis, Sildenafil, Vardenafil, Diphenoxylate, Simethicone, Cimetidine, Famotidine, Ranitidine, Ratinidine, cetrizin, Loratadine, Aspirin, Benzocaine, Dextrometorphan, Phenylpropanolamine, Pseudoephedrine, Cisapride, Domperidone, Metoclopramide, Acyclovir, Dioctylsulfosucc., Phenolphtalein, Almotriptan, Eletriptan, Ergotamine, Migea, Naratriptan, Rizatriptan, Sumatriptan, Zolmitriptan, Aluminum salts, Calcium salts, Ferro salts, Ag-salts, Zinc-salts, Amphotericin B, Miconazole, Triamcinolonacetonid, Melatonine, Phenobarbitol, Caffeine, Benzodiazepiner, Hydroxyzine, Meprobamate, Phenothiazine, Buclizine, Brometazine, Cinnarizine, Cyclizine, Difenhydramine, Dimenhydrinate, Buflomedil, Amphetamine, Caffeine, Ephedrine, Orlistat, Phenylephedrine, Phenylpropanolamin, Pseudoephedrine, Sibutramin, Ketoconazole, Nitroglycerin, Nystatin, Progesterone, Testosterone, Vitamin B12, Vitamin C, Vitamin A, Vitamin D, Vitamin E, Pilocarpin, Aluminumaminoacetat, Cimetidine, Esomeprazole, Famotidine, Lansoprazole, Magnesiumoxide, Nizatide and or Ratinidine.

The invention is suitable for increased or accelerated release of active agents selected among the group of dietary supplements, antiseptic agents, pH adjusting agents, anti-smoking agents, sweeteners, flavorings, aroma agents or drugs. Some of those will be described below.

The active agents to be used in connection with the present invention may be any substance desired to be released from the tablet. The active agents, for which a controlled and/or accelerated rate of release is desired, are primarily substances with a limited water-solubility, typically below 10 g/100 mL inclusive of substances which are totally water-insoluble. Examples are medicines, dietary supplements, oral compositions, anti-smoking agents, highly potent sweeteners, pH adjusting agents, flavorings etc.

Other active ingredients are, for instance, paracetamol, benzocaine, cinnarizine, menthol, carvone, caffeine, cyclizine hydrochloride, 1,8-cineol, nandrolone, miconazole, mystatine, nicotine, other quaternary ammonium compounds, vitamin E, vitamin A, vitamin D, glibenclamide or derivatives thereof, progesterone, acetylsalicylic acid, dimenhydrinate, cyclizine, metronidazole, sodium hydrogen carbonate, the active components from ginkgo, the active components from propolis, the active components from ginseng, methadone, oil of peppermint, salicylamide, hydrocortisone or astemizole.

Examples of active agents in the form of dietary supplements are for instance salts and compounds having the nutritive effect of vitamin B2 (riboflavin), B12, folinic acid, folic acid, niacine, biotine, poorly soluble glycerophosphates, amino acids, the vitamins A, D, E and K, minerals in the form of salts, complexes and compounds containing calcium, phosphorus, magnesium, iron, zinc, copper, iodine, manganese, chromium, selenium, molybdenum, potassium, sodium or cobalt.

Furthermore, reference is made to lists of nutritionists accepted by the authorities in different countries such as for instance US code of Federal Regulations, Title 21, Section 182.5013.182 5997 and 182.8013-182.8997.

Examples of active agents in the form of antiseptics are for instance salts and compounds of guanidine and biguanidine and the following types of substances with limited water-solubility: quaternary ammonium compounds (for instance ceramine, chloroxylenol, crystal violet, chloramine), aldehydes (for instance paraformaldehyde), compounds of dequaline, polynoxyline, phenols (for instance thymol, para chlorophenol, cresol) hexachlorophene, salicylic anilide compounds, triclosan, halogenes (iodine, iodophores, chloroamine, dichlorocyanuric acid salts), alcohols (3,4 dichlorobenzyl alcohol, benzyl alcohol, phenoxyethanol, phenylethanol), cf. furthermore Martindale, The Extra Pharmacopoeia, 28th edition, pages 547-578; metal salts, complexes and compounds with limited water-solubility, such as aluminum salts, (for instance aluminum potassium sulphate A1K(SO4)2,12H2O) and furthermore salts, complexes and compounds of boron, barium, strontium, iron, calcium, zinc, (zinc acetate, zinc chloride, zinc gluconate), copper (copper chloride, copper sulfate), lead, silver, magnesium, sodium, potassium, lithium, molybdenum, vanadium should be included.

Examples of active agents in the form of agents adjusting the pH in the oral cavity include for instance: acceptable acids, such as adipic acid, succinic acid, fumaric acid, or salts thereof or salts of citric acid, tartaric acid, malic acid, acetic acid, lactic acid, phosphoric acid and glutaric acid and acceptable bases, such as carbonates, hydrogen carbonates, phosphates, sulfates or oxides of sodium, potassium, ammonium, magnesium or calcium, especially magnesium and calcium.

Examples of active agents in the form of anti-smoking agents include for instance: nicotine, tobacco powder or silver salts, for instance silver acetate, silver carbonate and silver nitrate.

Further examples of active agents are medicines of any type.

Examples of active agents in the form of medicines include caffeine, salicylic acid, salicyl amide and related substances (acetylsalicylic acid, choline salicylate, magnesium salicylate, sodium salicylate), paracetamol, salts of pentazocine (pentazocine hydrochloride and pentazocinelactate), buprenorphine hydrochloride, codeine hydrochloride and codeine phosphate, morphine and morphine salts (hydrochloride, sulfate, tartrate), methadone hydrochloride, ketobemidone and salts of ketobemidone (hydrochloride), beta-blockers, (propranolol), calcium antagonists, verapamil hydrochloride, nifedinpine as well as suitable substances and salts thereof mentioned in Pharm. Int., November 85, pages 267-271, Barney H. Hunter and Robert L. Talbert, nitroglycerine, erythrityl tetranitrate, strychnine and salts thereof, lidocaine, tetracaine hydrochloride, etorphine hydrochloride, atropine, insulin, enzymes (for instance papain, trypsin, amyloglucosidase, glucoseoxidase, streptokinase, streptodornase, dextranase, alpha amylase), polypeptides (oxytocin, gonadorelin, (LH.RH), desmopressin acetate (DDAVP), isoxsuprine hydrochloride, ergotamine compounds, chloroquine (phosphate, sulfate), isosorbide, demoxytocin, heparin.

Other active ingredients include beta-lupeol, Letigen®, Sildenafil citrate and derivatives thereof.

Further examples of active ingredients include vitamins. Vitamins include A, B1, B2, B6, B12, Folinic acid, Folic acid, niacin, Pantothenic acid, biotine, C, D, E, K. Minerals include Calcium, phosphor, magnesium, iron, Zinc, Copper, Iod, Mangan, Crom, Selene, Molybden. Other active ingredients include: Q10®, enzymes. Natural drugs including Ginkgo Biloba, ginger, and fish oil.

Further examples of active ingredients include migraine drugs such as Serotonin antagonists: Sumatriptan, Zolmitriptan, Naratriptan, Rizatriptan, Eletriptan; nausea drugs such as Cyclizin, Cinnarizin, Dimenhydramin, Difenhydrinat; hay fever drugs such as Cetrizin, Loratidin, pain relief drugs such as Buprenorfin, Tramadol, oral disease drugs such as Miconazol, Amphotericin B, Triamcinolonaceton; and the drugs Cisaprid, Domperidon, Metoclopramid. In a preferred embodiment the invention relates to the release of Nicotine and its salts.

In an advantageous embodiment of the invention the active ingredient is selected from active ingredients for the throat selected from acetylcysteine, ambroxol, amylmetacresol, benzocaine, bisacodyl, bismuth sub salicylate, bromhexine, cetirizine, dextromethorphan hydrobromide, 2,4-dichlorobenzyl alcohol, doxylamine succinate, eucalyptus oil, flurbiprofen, glycerin, hexylresorcinol, lidocaine, menthol, myrrh, paracetamol, pectin, peppermint oil, phenol, phenylephrine, povidone-iodine, pseudoephedrine, ranitidine, simethicone, sodium docusate, spearmint, zinc, or any combination thereof; active ingredients for the gastrointestinal tract selected from alginate, atenolol, aspirin (acetylsalicylic acid), ampicillin, aminosalicylates, anhydrous citric acid, aspirin, bisacodyl, bismuth subsalicylate, bupropion, caffeine, calcium, calcium carbonate, cetirizine, cimetidine, cisapride, clarithromycin, desloratadine, dexlansoprazole, diphenhydramine HCl, diphenhydramine citrate, dimenhydrinate, docusate erythromycin, dopamine, esomeprazole, famotidine, fexofenadine HCl, guaifenesin, hydrotalcite, ibuprofen, ketoprofen, lactase enzyme, lansoprazole, loratadine, lorcaserin, loperamide, loperamide HCl, magnesium, magnesium carbonate, magnesium hydroxide, melatonin, methamphetamine HCl, metoclopramide, metronidazole, montelukast, mycostatin, naltrexone, naproxen, naproxen sodium, nizatidine, omeprazole, ondansetron, orlistat, pantoprazole, paracetamol (acetaminophen), pectin, phentermine HCl, polypodium leucotomos, prednisolone, prednisone, progesterone, propranolol, propantheline bromide, pseudoephedrine HCl, phentermine, rabeprazole, ranitidine, roflumilast, scopoloamine butyl hydroxide, simethicone, sodium, sodium bicarbonate, sodium docusate, sumatriptan, testosterone, tetracycline, topiramate, vitamin A, vitamin B, vitamin B12, vitamin C (ascorbic acid), vitamin D, and vitamin E, vitamin K, or any combination thereof, and active ingredients for buccal absorption selected from atenolol, baclofen, caffeine, carvedilol, chlorpheniramine, chlorpheniramine maleate, fluticasone propionate, maleate, desmopressin, diltiazem hydrochloride, doxylamine succinate, mycostatin, nicotine, nifedipine, nitroglycerin, omeprazole, ondansetron, oxymetazoline HCl, oxytocin, phenylephrine, piroxicam, prednisone, propranolol, salbutamol sulphate, scopoloamine butyl hydroxide, sumatriptan, triamcinolonacetonid, and any combination thereof.

In one aspect of the invention, the “tablet” is intended to mean a “fast disintegrating tablet” (“FDT”), or similar wording, such as “orally disintegrating tablet” (“ODT”). If not stated otherwise, if the tablet according to the invention is made as one module, contrary to two or more modules, then the tablet is intended to be an FDT tablet. If on the other hand, the tablet is made of more than one module, such as two modules, such additional module is intended to be a “lozenge” module or “chewing gum module”, which provides a longer disintegration time compared to the FDT module according to the invention. The combination of an “FDT” module and a “lozenge” module (or a “chewing gum module) contributes to another aspect of the invention. A “lozenge” module or “chewing gum module” according to the invention may also comprise elements from the “FDT” modules but is generally different in composition, providing an extended disintegration time.

The term “module” is generally intended to be composed of a composition of matter with substantially the same characteristics throughout the module. Hence, if two module are present, then the two modules are different in composition and generally have two different characteristics throughout each module. In the present context, if only one module is present, then this module is considered an FDT tablet. On the other hand, if two modules are present, then the tablet is composed of an FDT tablet or FDT tablet module fused with a lozenge tablet or lozenge module. The term “fused” is intended to mean that the tablet is gathered together by means of compression force. Usually, if two modules are present, the lozenge module is made as the first module and the FDT module is made as the second module. The tablet may be composed of more than two module. The lozenge module may in certain embodiments be replaced by a gum base module. In the present context, the invention provides an attractive bi-phasic delivery of masking, even if the delivery of nicotine is “single-phased”.

### EXAMPLES

### Example 1

Preparation of Dextrose Tablets

In a first step, dextrose was added to a mixing container. Binders, flavors, high-intensity sweeteners and optional other components were added to the container. In some of the comparative examples, binders were omitted. In some examples, one or more active ingredients were added and further specified in the specific examples below. The mixture was sieved and tumbled in a FUCHS Mixomat-A at approximately 25 rpm for 4 minutes. A processing aid was added and the mixture was tumbled at approximately 25 rpm for another 1 minute. Hereafter, the mixture was ready for tableting.

Dextrose applied according to the examples was C*dex 02001 commercially available from Cargill, unless otherwise indicated. In some examples, comparative grades were used. Particularly, binders applied were HPC and HPMC. HPC was available as Klucel Nutra D from Ashland. HPMC was available as Methocel 4KM from Dow. In some of the comparative examples, other binders were applied. When microcrystalline cellulose was applied as a comparative binder, it was Avicel PH-102 commercially available from Dupont. When maltodextrin was applied as a comparative binder, it was C*dry MD from Cargill.

The mixture was subsequently led to a standard tablet pressing machine (3090i, available from Fette GmbH) comprising dosing apparatus (P 3200 C, available from Fette GmbH, Germany) and pressed into tablets. Alternatively, a Riva Picoola Bi-layer DC-PL-015 was used. The tablets were pressed using a pressing pressure of 20-30 kN, unless otherwise indicated. There were 11 punches on the rotor, and the rotor speed used was 5 rpm. The individual tablets had a weight of approx. 1500 mg unless otherwise stated in the examples below. Punch used: 15.00 mm, circular, shallow concave, B tooling.

### Example 2

Composition of Dextrose Tablets with Different Content of Binder and in Presence of an Active Ingredient in the Same Amount

Dextrose tablets based on the procedure in Example 1 were made with the formulations outlined in the examples below. In all of the tablet examples, the amount of the various ingredients is given as % by weight of the tablet.

TABLE 1

| | Tablet Number | | | | |
|---|---|---|---|---|---|
| | 100-1A | 100-1B | 100-1C | 100-1D | 100-1E |
| | Content | Content | Content | Content | Content |
| Raw material name | [%] | [%] | [%] | [%] | [%] |
| Dextrose* | 96.0 | 95.0 | 93.0 | 91.0 | 86.0 |
| Binder** | — | 1.0 | 3.0 | 5.0 | 10.0 |
| Active ingredient*** | 0.1 | 0.1 | 0.1 | 0.1 | 0.1 |
| Flavors/high-intensity | 2.9 | 2.9 | 2.9 | 2.9 | 2.9 |
| sweeteners | | | | | |
| Processing aids | 1.0 | 1.0 | 1.0 | 1.0 | 1.0 |
| Total | 100 | 100 | 100 | 100 | 100 |

It was secured that the binders were thoroughly mixed into the dry mixture.
*Dextrose was C*dex 02001 commercially available from Cargill.
**The binder was HPC.
***The active ingredient was melatonin available from JiaHerb. The tablets were pressed at 30 kN.

Example 100-1A was a comparative example made in order to establish the effect in the absence of one or more binders.

The binder containing dextrose tablets of Table 1 were made again as tablet numbers 100-1B*, 100-1C*, 100-1D* and 100-1E* replacing HPC with HPMC as binder.

### Example 3

Composition of Dextrose Tablets with Same Content of Binder and in Presence of an Active Ingredient in Different Amounts

Dextrose tablets based on the procedure in Example 1 were made with the formulations outlined in the examples below. In all of the tablet examples, the amount of the various ingredients is given as % by weight of the tablet.

TABLE 2

| | Tablet Number | | | |
|---|---|---|---|---|
| | 100-2A | 100-2B | 100-2C | 100-2D |
| | Content | Content | Content | Content |
| Raw material name | [%] | [%] | [%] | [%] |
| Dextrose* | 91.8 | 71.8 | 51.8 | 31.8 |
| Binder** | 1.0 | 1.0 | 1.0 | 1.0 |
| Active ingredient*** | 5.0 | 25.0 | 45.0 | 65.0 |
| Flavors/high-intensity | 1.2 | 1.2 | 1.2 | 1.2 |
| sweeteners | | | | |
| Processing aids | 1.0 | 1.0 | 1.0 | 1.0 |
| Total | 100 | 100 | 100 | 100 |

It was secured that the binders were thoroughly mixed into the dry mixture.
*Dextrose was C*dex 02001 commercially available from Cargill.
**The binder was HPC.
***The active ingredient was calcium carbonate available from Nutrigranulation. The tablets were pressed at 30 kN.

Example 100-2D was a comparative example made in order to establish the effect of a content of dextrose below 50% by weight of the tablet.

The dextrose tablets of Table 2 were made again as tablet numbers 100-2A*, 100-2B*, 100-2C*, and 100-2D* replacing HPC with HPMC as binder.

### Example 4

Composition of Dextrose Tablets with Different Content of Binder and in Presence of an Active Ingredient in Substantially the Same Amount

Dextrose tablets based on the procedure in Example 1 were made with the formulations outlined in the examples below, here including an active ingredient in an amount of 675 mg for 100-2E and 100-2C, and 630 mg for 100-2F and 100-2G. In all of the tablet examples, the amount of the various ingredients is given as % by weight of the tablet.

TABLE 3

| | Tablet Number | | | |
|---|---|---|---|---|
| | 100-2E | 100-2C | 100-2F | 100-2G |
| | Content | Content | Content | Content |
| Raw material name | [%] | [%] | [%] | [%] |
| Dextrose* | 52.8 | 51.8 | 52.8 | 50.8 |
| Binder** | — | 1.0 | 3.0 | 5.0 |
| Active ingredient*** | 45.0 | 45.0 | 42.0 | 42.0 |
| Flavors/high-intensity sweeteners | 1.2 | 1.2 | 1.2 | 1.2 |
| Processing aids | 1.0 | 1.0 | 1.0 | 1.0 |
| Total | 100 | 100 | 100 | 100 |

It was secured that the binders were thoroughly mixed into the dry mixture.
*Dextrose was C*dex 02001 commercially available from Cargill.
**The binder was HPC.
***The active ingredient was calcium carbonate available from Nutrigranulation. The tablets were pressed at 30 kN.

Example 100-2E was a comparative example made in order to establish the effect in the absence of one or more binders.

The dextrose tablets 100-2F and 100-2G of Table 3 were made again as tablet numbers 100-2F*, and 100-2G* replacing HPC with HPMC as binder.

It is noted that the difference in amounts of 42 respectively 45 percent by weight of active ingredients was considered insignificant.

### Example 5

Composition of Dextrose Tablets with Different Content of Binder and in Presence of an Active Ingredient in the Same Amount

Dextrose tablets based on the procedure in Example 1 were made with the formulations outlined in the examples below, here including an active ingredient in an amount of 630 mg. In all of the tablet examples, the amount of the various ingredients is given as % by weight of the tablet.

TABLE 4

| | Tablet Number | | | |
|---|---|---|---|---|
| | 100-2H | 100-2I | 100-2J | 100-2K |
| | Content | Content | Content | Content |
| Raw material name | [%] | [%] | [%] | [%] |
| Dextrose* | 52.8 | 50.8 | 50.8 | 45.8 |
| MCC binder | 3.0 | 5.0 | — | — |
| Maltodextrin binder | — | — | 5.0 | 10.0 |
| Active ingredient*** | 42.0 | 42.0 | 42.0 | 42.0 |
| Flavors/high-intensity sweeteners | 1.2 | 1.2 | 1.2 | 1.2 |
| Processing aids | 1.0 | 1.0 | 1.0 | 1.0 |
| Total | 100 | 100 | 100 | 100 |

It was secured that the binders were thoroughly mixed into the dry mixture.
*Dextrose was C*dex 02001 commercially available from Cargill.
***The active ingredient was calcium carbonate available from Nutrigranulation. The tablets were pressed at 30 kN.

### Example 6

Evaluations of Tablets

For each version of the tablets, a breaking point test, a friability test and a dissolution time measurement were performed. For measuring breaking point, a PTB 311 from Pharma Test was used.

The friability test was made in accordance to European Pharmacopoeia 9.1, test method 2.9.7. by using a pharmaceutical friability-tester PTF 10E from Pharma Test.

To test dissolution time, the following method was used. 15 mL of 0.02 M potassium dihydrogen phosphate-buffer (pH adjusted to 7.4) is added to 50 mL of water in a measuring tube with a screw cap. The tablet is inserted in the measuring tube and the screw cap is fastened. The measuring tube is fixated horizontally. The measuring tube is vibrated at about 110 RPM such that the tablet can move back and forth in the measuring tube. The measuring tube is vibrated until the tablet or module thereof in question is completely dissolved and the time of vibration is noted as the dissolution time.

### Example 7

Sensorial Evaluation of Tablets

Sensoric tests were performed to reveal very important characteristics and properties of the tablets. These sensoric parameters are important as indicators of the structure of the tablet composition. The test set-up was composed of 8 test persons in a test panel. All of the test persons were healthy individuals appointed on an objective basis according to specified requirements. The sensory analysis was performed according to ISO 4121-2003 in testing conditions following ISO 8589. The result is an average of the results of the 8 individuals.

The test persons gave a rating from “+” to “+++++”, where “+” is poor and “+++++” is excellent. “0” indicated that it was not tested.

Six different parameters were tested in a test panel:

“Ease of chewing into liquid”—the impression of the tablet when placed in the mouth and chewed with respect to easiness of chewing the product into liquid. The criteria is that upon completion, there is no sense of particles in the mouth, and the powder of the tablet has dissolved into liquid.

“Liquid feeling”—the impression of the tablet when placed in the mouth and chewed with respect to the sense of liquid in the mouth. For instance, if more liquid is sensed during and/or after chewing, then the score is high.

“Mouthfeel”—the overall impression of the tablet during chewing with respect to mouthfeel, including melting and tacking sensations. A high scoring mouthfeel is associated with a clean liquid (no sense of particles), no tablet residuals sticking in teeth and a creamy feeling (higher viscosity than water). On the contrary, a low scoring mouthfeel is associated with sense of particles in the liquid (incomplete dissolution), tablet residuals sticking in the teeth and a watery feeling of the liquid.

“Overall taste”—the overall impression of the taste of the tablet during chewing. For instance, if the taste was decreasing rapidly, a very low rating was given.

“Overall sweetness”—the overall impression of the sweetness of the tablet during chewing. For instance, if the sweetness was decreasing rapidly, a very low rating was given.

“Overall sourness”—the overall impression of the sourness of the tablet during chewing. For instance, if the sourness was decreasing rapidly, a very low rating was given.

### Example 8

Results of Composition of Dextrose Tablets with Different Content of Binder and in Presence of an Active Ingredient in the Same Amount

TABLE 5

| | Tablet Number | | | | |
|---|---|---|---|---|---|
| | 100-1A | 100-1B | 100-1C | 100-1D | 100-1E |
| Ease of chewing | +++++ | +++++ | +++++ | ++++ | ++ |
| into liquid | | | | | |
| Mouthfeel | +++++ | +++++ | +++++ | ++++ | +++ |
| Comments | Easy, no | Easy, no | Easy, no | Easy, no | Easy, no |
| | sticking, no | sticking, no | sticking, no | sticking, no | sticking, no |
| | grease or | grittiness | grittiness | grittiness | grittiness |
| | grittiness | | | | |
| Friability | 1.73 | 0.87 | 0.47 | 0.27 | 0.20 |

Tested in compliance with Examples 6 and 7.

Generally, the results reveal that HPC was a superior binder with resulting low friability as a function of the level of binder. However, friability was clearly inferior when no binder was added. When using a high level of binder, the sensorial parameters were lower than if less binder was applied, although friability was improved. Specifically, it is contemplated that above 5% of binder is unsuitable for the dextrose tablets.

### Example 9

Results of Composition of Dextrose Tablets with Same Content of Binder and in Presence of an Active Ingredient in Different Amounts

TABLE 6

| | Tablet Number | | | |
|---|---|---|---|---|
| | 100-2A | 100-2B | 100-2C | 100-2D |
| Ease of chewing into | +++++ | +++++ | ++++ | ++ |
| liquid | | | | |
| Mouthfeel | +++++ | ++++ | ++++ | +++ |
| Comments | Good mouthfeel, | Good mouthfeel, | Good mouthfeel, | Ok mouthfeel, |
| | almost no sense of | liquid turns slightly | liquid turns slightly | liquid turns too |
| | coarseness | powdery | powdery | powdery |
| Friability | 1.20 | 0.86 | 0.47 | 0.47 |

Tested in compliance with Examples 6 and 7.

Generally, the results reveal that the level of active ingredients had an impact on the system. When using a high level of active ingredients and thereby a low level of dextrose, the products were not sensorially acceptable. It is contemplated that below 50% of dextrose is unsuitable for the dextrose tablets. It was not expected that a high amount of active ingredients as used in a number of the examples were possible to add in the dextrose tablets without compromising the suitability of the dextrose tablets. Particularly, it was a surprise that examples 100-2B and 100-2C revealed beneficial sensorial properties even with the high amount of actives used. With respect to example 100-2D, the amount of actives compromised the sensorial properties of the dextrose tablets.

### Example 10

Results of Composition of Dextrose Tablets with Different Content of Binder and in Presence of an Active Ingredient in Substantially the Same Amount

TABLE 7

| | Tablet Number | | | |
|---|---|---|---|---|
| | 100-2E | 100-2C | 100-2F | 100-2G |
| Ease of chewing into | ++++ | ++++ | ++++ | ++++ |
| liquid | | | | |
| Mouthfeel | ++++ | ++++ | ++++ | +++ |
| Comments | Good mouthfeel, | Good mouthfeel, | Good mouthfeel, | Good mouthfeel, |
| | liquid turns slightly | liquid turns slightly | liquid turns slightly | liquid turns slightly |
| | powdery | powdery | powdery | powdery |
| Friability | 1.00 | 0.47 | 0.47 | 0.13 |

Tested in compliance with Examples 6 and 7.

Generally, the results reveal that HPC was a superior binder with resulting low friability as a function of the level of binder.

### Example 11

Results of Composition of Dextrose Tablets with Different Content of Binder and in Presence of an Active Ingredient in the Same Amount

TABLE 8

| | Tablet Number | | | |
|---|---|---|---|---|
| | 100-2H | 100-2I | 100-2J | 100-2K |
| Ease of chewing into | +++ | ++ | +++ | ++ |
| liquid | | | | |
| Mouthfeel | ++ | ++ | ++ | + |
| Comments | Greasy coat | Greasy coat | Greasy coat | Greasy coat |
| | feeling, stickiness | feeling, stickiness | feeling, stickiness | feeling, stickiness |
| Friability | 0.53 | 0.60 | 0.73 | 0.73 |

Tested in compliance with Examples 6 and 7.

Generally, the results reveal that MCC and maltodextrin were poor binders and the sensorial parameters were poor. HPC was clearly a more appropriate binder providing a superior mouthfeel.

### Example 12

Composition of Dextrose Tablets with Different Active Ingredients Focused on Energy

Dextrose tablets based on the procedure in Example 1 were made with the formulations outlined in the examples below, here including caffeine in an amount of 100 mg and optionally vitamin B premix in an amount of 15 mg as active ingredient. In all of the tablet examples, the amount of the various ingredients is given as % by weight of the tablet.

TABLE 9

| | Tablet Number | | | |
|---|---|---|---|---|
| | 100-3A | 100-3B | 100-3C | 100-3D |
| | Content | Content | Content | Content |
| Raw material name | [%] | [%] | [%] | [%] |
| Dextrose* | 89.8 | 88.8 | 89.8 | 88.8 |
| HPC binder | 1.0 | 1.0 | — | — |
| HPMC binder | — | — | 1.0 | 1.0 |
| Caffeine*** | 6.8 | 6.8 | 6.8 | 6.8 |
| Vitamin B**** | — | 1.0 | — | 1.0 |
| Flavors/high-intensity | 1.4 | 1.4 | 1.4 | 1.4 |
| sweeteners | | | | |
| Processing aids | 1.0 | 1.0 | 1.0 | 1.0 |
| Total | 100 | 100 | 100 | 100 |

It was secured that the binders were thoroughly mixed into the dry mixture.
*Dextrose was C*dex 02001 commercially available from Cargill.
***Caffeine available from Siegfried,
****Vitamin B premix available from DSM. The tablets were pressed at 27 kN.

### Example 13

Composition of Dextrose Tablets with Different Active Ingredients Focused on Immune Stimulants

Dextrose tablets based on the procedure in Example 1 were made with the formulations outlined in the examples below, here including vitamin C in an amount of 500 mg or a herbal blend with vitamin C and other vitamins/minerals in an amount of 450 mg as active ingredient. In all of the tablet examples, the amount of the various ingredients is given as % by weight of the tablet.

TABLE 10

| | Tablet Number | | | |
|---|---|---|---|---|
| | 100-4A | 100-4B | 100-4C | 100-4D |
| | Content | Content | Content | Content |
| Raw material name | [%] | [%] | [%] | [%] |
| Dextrose* | 62.8 | 66.1 | 62.8 | 66.1 |
| HPC binder | 1.0 | 1.0 | — | — |
| HPMC binder | — | — | 1.0 | 1.0 |
| Vitamin C*** | 33.3 | — | 33.3 | — |
| Herbal blend with vitamins* * * * | — | 30.0 | — | 30.0 |
| Flavors/high-intensity sweeteners | 1.9 | 1.9 | 1.9 | 1.9 |
| Processing aids | 1.0 | 1.0 | 1.0 | 1.0 |
| Total | 100 | 100 | 100 | 100 |

It was secured that the binders were thoroughly mixed into the dry mixture.
*Dextrose was C*dex 02001 commercially available from Cargill.
***Vitamin C available from DSM,
****Herbal blend with vitamins available from DSM. The tablets were pressed at 70 kN.

### Example 14

Composition of Dextrose Tablets with Different Active Ingredients Focused on Cough and Cold Formulations

Dextrose tablets based on the procedure in Example 1 were made with the formulations outlined in the examples below, here including active ingredients suitable for treating cough and cold symptoms. In all of these tablet examples, the total tablet weight is 1750 mg, and the amount of the various ingredients is given as mg.

TABLE 11
It was secured that the binders were thoroughly mixed into the dry mixture.

| | Tablet Number | | | | | |
|---|---|---|---|---|---|---|
| | 100-5A | 100-5B | 100-5C | 100-5D | 100-5E | 100-5F |
| | Content | Content | Content | Content | Content | Content |
| Raw material name | [mg] | [mg] | [mg] | [mg] | [mg] | [mg] |
| Dextrose* | 1325 | 1310 | 1000 | — | — | — |
| Dextrose** | — | — | — | 1325 | 1310 | 1000 |
| HPC binder | 35 | 35 | 35 | 35 | 35 | 35 |
| Acetominophen*** | 250 | 250 | 250 | 250 | 250 | 250 |
| Dextromethorphan**** | — | 10 | 10 | — | 10 | 10 |
| Phenylephrine***** | — | 5 | 5 | — | 5 | 5 |
| Mannitol (Pearlitol DC300) | — | — | 310 | — | — | 310 |
| Flavors/high-intensity | 123 | 123 | 123 | 123 | 123 | 123 |
| sweeteners/fruit acids | | | | | | |
| Processing aids | 17 | 17 | 17 | 17 | 17 | 17 |
| Total | 1750 | 1750 | 1750 | 1750 | 1750 | 1750 |

*Dextrose was C*dex 02001 commercially available from Cargill.
**Dextrose was Emdex commercially available from JRS Pharma.
***Acetominophen available from Mallinckrodt,
****Dextromethorphan HBr available from LGM Pharma,
*****Phenylephrine HCl available from Siegfried.
The tablets were pressed at 70 kN.

### Example 15

Results of Comparison of Dextrose Tablet 100-4B with a Commercially Available Product Containing Immune Stimulants

TABLE 12

| | Tablet Number | |
|---|---|---|
| | 100-4B | Airborne |
| Ease of chewing into liquid | +++++ | +++ |
| Mouthfeel | ++++ | ++ |
| Comments | Good mouthfeel, almost | Crumble mouthfeel, |
| | no sandy mouth-feel | sandy |
| | | mouthfeel |

Tested in compliance with Examples 6 and 7.

Generally, the results reveal that dextrose tablet 100-4B was providing a superior mouthfeel and liquifying sensation compared to the commercially available product Airborne which contains the same type of actives as dextrose tablet 100-4B.

1-99. (canceled)

100. An oral chewable tablet suitable for improved mouthfeel, comprising dextrose in an amount from 50 to 95% by weight of the tablet;
one or more active ingredients; and
one or more binders, wherein
a ratio between the one or more binders and the dextrose is from 1:250 to 1:8.

101. The oral chewable tablet according to claim 100, wherein the ratio between the one or more binders and the dextrose is from 1:200 to 1:10.

102. The oral chewable tablet according to claim 100, wherein the ratio between the one or more binders and the dextrose is from 1:250 to 1:25 if the tablet comprises the dextrose in an amount from 70 to 95% by weight of the tablet.

103. The oral chewable tablet according to claim 100, wherein the ratio between the one or more binders and the dextrose is from 1:100 to 1:8 if the tablet comprises the dextrose in an amount from 50 to 70% by weight of the tablet.

104. The oral chewable tablet according to claim 100, wherein the ratio between the one or more binders and the one or more active ingredients is from 1:75 to 1:8.

105. The oral chewable tablet according to claim 100, wherein the dextrose is present in an amount from 70 to 95% by weight of the tablet.

106. The oral chewable tablet according to claim 100, wherein the oral chewable tablet is consisting essentially of the dextrose, the one or more active ingredients and the one or more binders, except for auxiliary ingredients present up to about 5% by weight of the tablet.

107. The oral chewable tablet according to claim 100, wherein the dextrose comprises at least 90% dextrose equivalents calculated on a dry basis.

108. The oral chewable tablet according to claim 100, wherein the dextrose comprises 100% dextrose equivalents calculated on a dry basis.

109. The oral chewable tablet according to claim 100, wherein the dextrose comprises at least 30% by weight of particles in a range of 100 to 500 microns.

110. The oral chewable tablet according to claim 100, wherein the dextrose comprises at least 80% by weight of particles below 500 microns.

111. The oral chewable tablet according to claim 100, wherein the one or more binders is present in an amount of 0.4 to 5% by weight of the tablet.

112. The oral chewable tablet according to claim 100, wherein the one or more binders is selected from the group consisting of hydroxypropyl cellulose (HPC), hydroxypropyl methylcellulose (HPMC), and combinations thereof.

113. The oral chewable tablet according to claim 100, wherein the one or more active ingredients is present in an amount of 5 to 50% by weight of the tablet.

114. The oral chewable tablet according to claim 100, wherein the one or more active ingredients comprises an immune supporting active ingredient.

115. The oral chewable tablet according to claim 100, wherein the one or more active ingredients comprises a mixture of vitamins, minerals, and herbals.

116. The oral chewable tablet according to claim 100, wherein the one or more active ingredients comprise an active pharmaceutical ingredient.

117. The oral chewable tablet according to claim 100, wherein the oral chewable tablet comprises one or more disintegrants operable to disintegrate the tablet within a period of 2 minutes or less in contact with oral saliva.

118. The oral chewable tablet according to claim 100, wherein the oral chewable tablet comprises one or more disintegrants selected from the group consisting of sodium croscarmellose, crospovidone, sodium starch glycolate, and combinations thereof.

119. The oral chewable tablet according to claim 100, wherein the one or more active ingredients is present in an amount of 1 to 1000 mg.

120. The oral chewable tablet according to claim 100, wherein the oral chewable tablet comprises a further tablet module that is different in composition.
